SEQUENCE LISTING (10 sequences)

SEQ ID NO: 1 - protein, 5 aa, Homo sapiens
SYWMS

SEQ ID NO: 2 - protein, 17 aa, Homo sapiens
SITSYGSFTYYADSVKG

SEQ ID NO: 3 - protein, 8 aa, Homo sapiens
NMYTHFDS

SEQ ID NO: 4 - protein, 13 aa, Homo sapiens
SGSSSNIGSNSVS

SEQ ID NO: 5 - protein, 7 aa, Homo sapiens
DNSKRPS

SEQ ID NO: 6 - protein, 11 aa, Homo sapiens
QSRDTYGYYWV

SEQ ID NO: 7 - protein, 117 aa, Homo sapiens
QVELVESGGGLVQPGGSLRLSCAASGFTFTSYWMSWVRQAPGKGLELVSSITSYGSFTYYADSVKGRFTISRDNSKNTLYLQMNSLRAEDTAVYYCARNMYTHFDSWGQGTLVTVSS

SEQ ID NO: 8 - protein, 111 aa, Homo sapiens
DIVLTQPPSVSGAPGQRVTISCSGSSSNIGSNSVSWYQQLPGTAPKLLIYDNSKRPSGVPDRFSGSKSGTSASLAITGLQSEDEADYYCQSRDTYGYYWVFGGGTKLTVLG

SEQ ID NO: 9 - protein, 447 aa, Homo sapiens
QVELVESGGGLVQPGGSLRLSCAASGFTFTSYWMSWVRQAPGKGLELVSSITSYGSFTYYADSVKGRFTISRDNSKNTLYLQMNSLRAEDTAVYYCARNMYTHFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 216 aa, Homo sapiens
DIVLTQPPSVSGAPGQRVTISCSGSSSNIGSNSVSWYQQLPGTAPKLLIYDNSKRPSGVPDRFSGSKSGTSASLAITGLQSEDEADYYCQSRDTYGYYWVFGGGTKLTVLGQPKAAPSVTLFPPSSEELQANKATLVCLISDFYPGAVTVAWKGDSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2017/0088609 A1
(43) Pub. Date: Mar. 30, 2017

(54) ANTICANCER COMBINATION THERAPY

(71) Applicant: Boehringer Ingelheim International GmbH, Ingelheim am Rhein (DE)

(72) Inventors: Flavio SOLCA, Vienna (AT); Ulrike WEYER-CZERNILOFSKY, Baden (AT); Jin Kyung RHO, Seoul (KR); Jae Cheol LEE, Seoul (KR)

(21) Appl. No.: 15/276,869

(22) Filed: Sep. 27, 2016

(30) Foreign Application Priority Data
Sep. 28, 2015 (EP) 15187238.9

(51) Int. Cl. C07K 16/22; A61K 31/506; A61K 39/395; A61K 31/519

(52) U.S. Cl. CPC C07K 16/22; A61K 31/519; A61K 31/506; A61K 39/39558; C07K 2317/565; C07K 2317/51; C07K 2317/515; C07K 2317/76

(57) ABSTRACT

The invention describes anti-cancer therapies comprising using a 3G-EGFR inhibitor and an anti-IGF antibody, each as described herein.

### FIELD OF THE INVENTION

The invention relates to anti-cancer therapies comprising a 3G-EGFR inhibitor and an anti-IGF antibody.

### BACKGROUND OF THE INVENTION

Epidermal growth factor receptor (EGFR) tyrosine kinase inhibitors (TKI) have marked a new era in the treatment of advanced non-small cell lung cancer (NSCLC). Over the last decade, EGFR TKIs established a remarkable therapeutic benefit in the patients with advanced NSCLC harboring EGFR activating mutations [1-7]. Unfortunately, however, efficacy of 1^{st} generation EGFR TKIs gefitinib and erlotinib is ultimately limited by inevitable development of acquired resistance (AR) after median of 10 to 12 months [8-11]. T790M is known to be the most common mechanism of AR observed in approximately 50 to 60% of patients. In this gatekeeper mutation, a well conserved threonine at codon 790 in exon 20 of EGFR undergoes substitution to bulkier methionine, which leads to steric hindrance of erlotinib binding in the ATP-kinase-binding pocket [8]. 2^{nd} generation EGFR TKIs, including afatinib (BIBW2992) and dacomitinib (PF299804), effectively inhibit T790M-containing cell lines in several preclinical models. In addition, mutant selective, 3^{rd} generation EGFR TKI, which comprises the irreversible pyrimidine-based WZ 4002 (compound 2 as described herein below) and newer compounds, i.e. AZD9291, CO1686, and HM61713 (compound 1 as described herein below) [12], have been developed. Strikingly, recent preclinical and preliminary clinical data demonstrated an outstanding clinical efficacy of 3^{rd} generation EGFR TKIs in patients with advanced NSCLC harboring T790M [13-18]. However, despite 3^{rd} generation EGFR TKIs emerging at the forefront in the treatment of EGFR mutant NSCLC, in practice patients finally experience disease progression regardless of clinical responses. It suggests the successive evolvement of AR beyond T790M, that is, 3^{rd} generation EGFR TKIs alone, are insufficient to control the disease.

Hence there is still a need for additional treatment options for patients with cancer and, in particular, solid tumors. There is also a need for additional treatment options for patients with lung cancer, such as NSCLC. One such method of boosting effectiveness of EGFR inhibitors in vivo is by interfering with the formation of the vasculature of the tumours and/or by dually targeting other proteins implicated in disease progression of cancer patients, i.e. a dual pathway blockade which can abrogate this resistance.

### SUMMARY OF THE INVENTION

It is thus an object of the invention to provide combination treatments/methods of combination treatment allowing for such dual (combined) pathway blockade and providing certain advantages compared to treatments/methods of treatment currently used and/or known in the prior art. These advantages may include in vivo efficacy (e.g. improved clinical response, e.g. extend of the response, duration of response, response rate, stabilization rate, duration of stabilization, time to disease progression, progression free survival (PFS) and/or overall survival (OS), later occurrence of resistance and the like), safe and well tolerated administration and reduced frequency of adverse events, in particular reduced frequency of the typical EGFR-mediated adverse events.

In this context, the IGF signaling pathway has been implicated in AR to EGFR TKIs among various non-T790M mechanisms. Combination with a TKI targeting the IGF-1/insulin receptor led to re-sensitization to compound 2 in mutant-EGFR, T790M-negative NSCLC [25], suggesting that by-pass signaling via the IGF pathway can arise as an EGFR-independent mechanism of resistance, occurring alternatively to T790M mutation.

The inventors of the current application, surprisingly, discovered that resistance of NSCLC to a 3^{rd} generation EGFR TKI via IGF pathway signaling can also occur in NSCLC harboring the T790M mutation, and that by-pass signaling is dependent on the presence of active IGF ligands. The use of a selective anti-IGF1/2 monoclonal antibody (preferably anti-IGF1/2 monoclonal antibody “BI-IGF” as disclosed herein) in a combination with a mutant-selective 3^{rd} generation EGFR TKI (e.g. WZ4002=compound 2 or HM61713=compound 1) is able to overcome resistance to a mutant-selective 3^{rd} generation EGFR TKI monotherapy. This study is the first to provide evidence that ligand-induced IGF-1R activation can be associated with acquired resistance to 3^{rd} generation EGFR TKI that develops after failure of prior treatment with 1^{st} generation EGFR TKIs due to T790M mutation, and therefore, is of particular clinical significance for this patient population. Thus, the invention relates to methods for the treatment and/or prevention of oncological or hyperproliferative diseases, in particular cancer, comprising the combined administration of a mutant-selective 3^{rd} generation EGFR TKI (referred to herein as “3G-EGFR inhibitor”) and an anti-IGF antibody, as well as to medical uses, to uses, to pharmaceutical compositions or combinations and kits comprising such active ingredients.

Further, the invention relates to anti-cancer therapies comprising using a 3G-EGFR inhibitor and an anti-IGF antibody, each as described herein, in combination.

For the treatment of diseases of oncological nature, a large number of anticancer agents (including target-specific and non-target-specific anticancer agents) have already been suggested, which can be used as monotherapy or as combination therapy involving more than one agent (e.g. dual or triple combination therapy) and/or which may be combined with radiotherapy (e.g. irradiation treatment), radio-immunotherapy and/or surgery.

Even if the concept of combining several therapeutic agents or therapies has already been suggested, and although various combination therapies are under investigation and in clinical trials, there is still a need for new and efficient therapies of cancer diseases, which show advantages over standard therapies, such as for example better treatment outcome, beneficial effects, superior efficacy and/or improved tolerability, such as e.g. reduced side effects of the combined treatment.

It is a purpose of the present invention to provide combination therapies with the active agents described herein for treating or controlling various malignancies (e.g. based on cooperative, complementary, interactive or improving effects of the active components involved in combination).

Thus, in one aspect, the invention provides a method of treating and/or preventing an oncological or hyperproliferative disease, in particular cancer, comprising administering to a patient in need thereof a therapeutically effective amount of a 3G-EGFR inhibitor and an anti-IGF antibody, each as described herein.

Such a combined treatment may be given as a free combination of the substances or in the form of a fixed combination, including kit-of-parts.

In another aspect, the invention refers to a combination of a 3G-EGFR inhibitor and an anti-IGF antibody, each as described herein, particularly for use in a method of treating and/or preventing an oncological or hyperproliferative disease, in particular a cancer disease e.g. as described herein, said method comprising administering to a patient in need thereof a therapeutically effective amount of the combination.

In another aspect, the invention refers to a 3G-EGFR inhibitor as described herein for use in a method of treating and/or preventing an oncological or hyperproliferative disease, in particular cancer, said method comprising administering the 3G-EGFR inhibitor in combination with an anti-IGF antibody as described herein to the patient in need thereof.

In another aspect, the invention refers to an anti-IGF antibody as described herein for use in a method of treating and/or preventing an oncological or hyperproliferative disease, in particular cancer, said method comprising administering the anti-IGF antibody in combination with a 3G-EGFR inhibitor as described herein to the patient in need thereof.

In another aspect, the invention refers to a kit including a first pharmaceutical composition or dosage form which comprises a 3G-EGFR inhibitor as described herein, and a second pharmaceutical composition or dosage form which comprises an anti-IGF antibody as described herein.

In another aspect, the invention refers to a pharmaceutical composition containing a 3G-EGFR inhibitor as described herein, an anti-IGF antibody as described herein, and, optionally, one or more pharmaceutically acceptable carriers, excipients and/or vehicles.

In another aspect, the invention refers to the use of a 3G-EGFR inhibitor as described herein for preparing a pharmaceutical composition for treating and/or preventing an oncological or hyperproliferative disease, in particular cancer (such as e.g. a cancer disease as described herein), wherein the 3G-EGFR inhibitor is to be used in combination with an anti-IGF antibody as described herein.

In another aspect, the invention refers to the use of an anti-IGF antibody as described herein for preparing a pharmaceutical composition for treating and/or preventing an oncological or hyperproliferative disease, in particular cancer (such as e.g. a cancer disease as described herein), wherein the anti-IGF antibody is to be used in combination with a 3G-EGFR inhibitor as described herein.

In another aspect, the invention refers to the use of a 3G-EGFR inhibitor and an anti-IGF antibody, each as described herein, for preparing a pharmaceutical composition for treating and/or preventing an oncological or hyperproliferative disease, in particular cancer (such as e.g. a cancer disease as described herein).

In another aspect, the invention refers to a combination, composition or kit according to the invention comprising, consisting or consisting essentially of a 3G-EGFR inhibitor and an anti-IGF antibody, each as described herein, e.g. for treating and/or preventing an oncological or hyperproliferative disease, in particular cancer (e.g. a cancer disease as described herein), optionally in combination with one or more other therapeutic agents.

In another aspect, the invention refers to a combination or kit comprising
a) a 3G-EGFR inhibitor and optionally one or more pharmaceutically acceptable carriers, excipients and/or vehicles,
b) an anti-IGF antibody and optionally one or more pharmaceutically acceptable carriers, excipients and/or vehicles,

and optionally a package insert comprising printed instructions for simultaneous, concurrent, sequential, successive, alternate or separate use in the treatment and/or prevention of an oncological or hyperproliferative disease, in particular cancer, optionally in combination with one or more other therapeutic agents, in a patient in need thereof.

In another aspect, the invention refers to a combination, composition or kit according to the invention optionally further comprising one or more other therapeutic agents.

In another aspect, the invention refers to a method or use according to the invention optionally further comprising administering or involving one or more other therapeutic agents.

In all aspects of the invention as described herein the 3G-EGFR inhibitor can be used in free form or in the form of a pharmaceutically acceptable salt.

### BRIEF DESCRIPTION OF THE FIGURES

FIG. 1 shows the phosphorylation status of multiple receptor tyrosine kinases (RTK) in PC-9 and PC-9/GR/WR cells, assessed by Phospho-RTK array analysis of cell lysates.

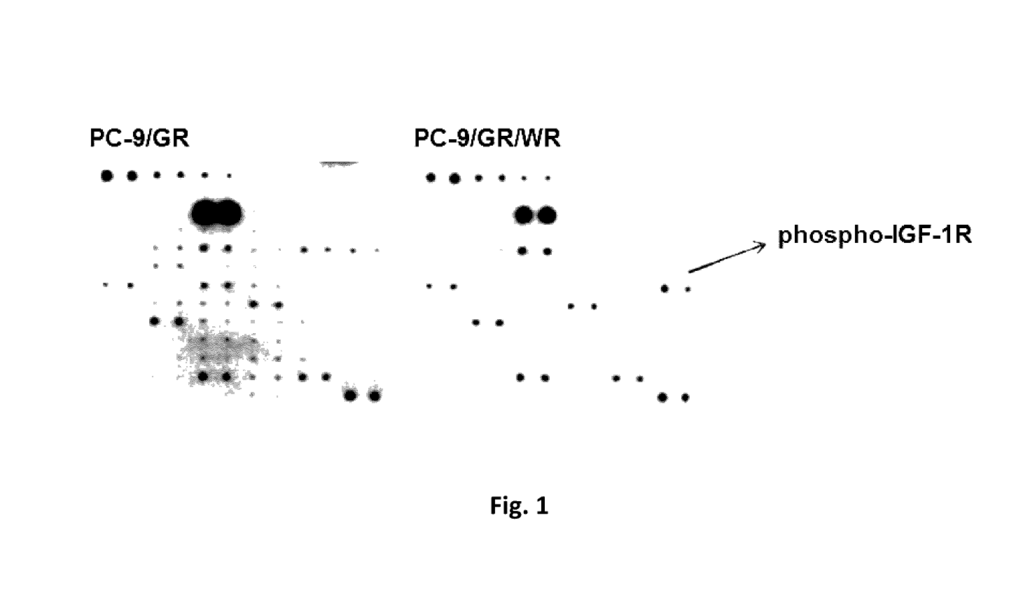

FIG. 2 shows the effect of WZ4002 (compound 2) on the proliferation of PC-9/GR and PC-9/GR/WR cells, and the effect of the combination of WZ4002 with the IGF1/2 neutralizing antibody BI-IGF on the proliferation of PC-9/GR/WR cells.

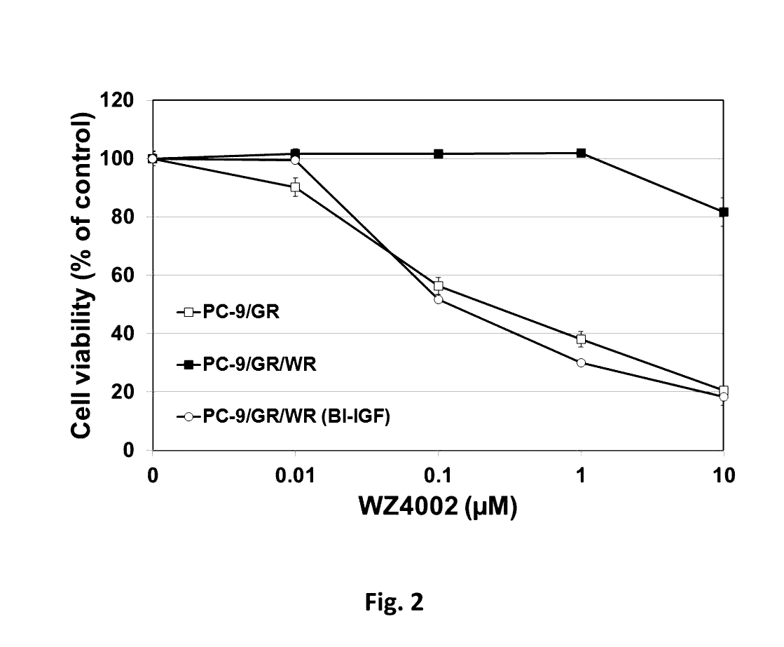

FIG. 3 shows the effect of treatment with WZ4002 and BI-IGF, alone and in combination, on the phosphorylation status of EGFR, IGF1R, and downstream signaling molecules, and on levels of apoptotic markers in PC-9/GR/WR cells in vitro.

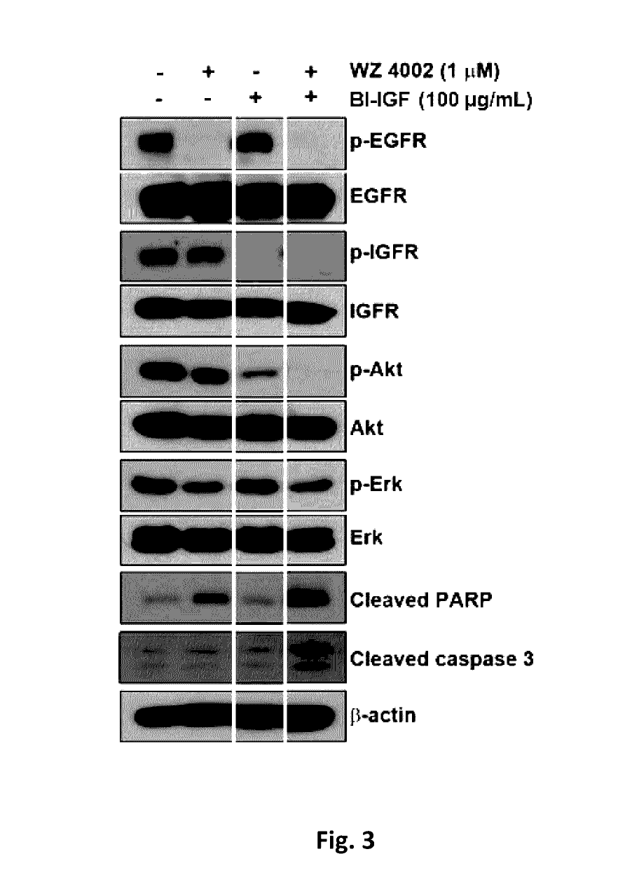

FIG. 4 shows the in vivo anti-tumor effect of WZ4002 and BI-IGF, alone and in combination, on the growth of PC-9/GR/WR tumour xenografts in immunodeficient mice.

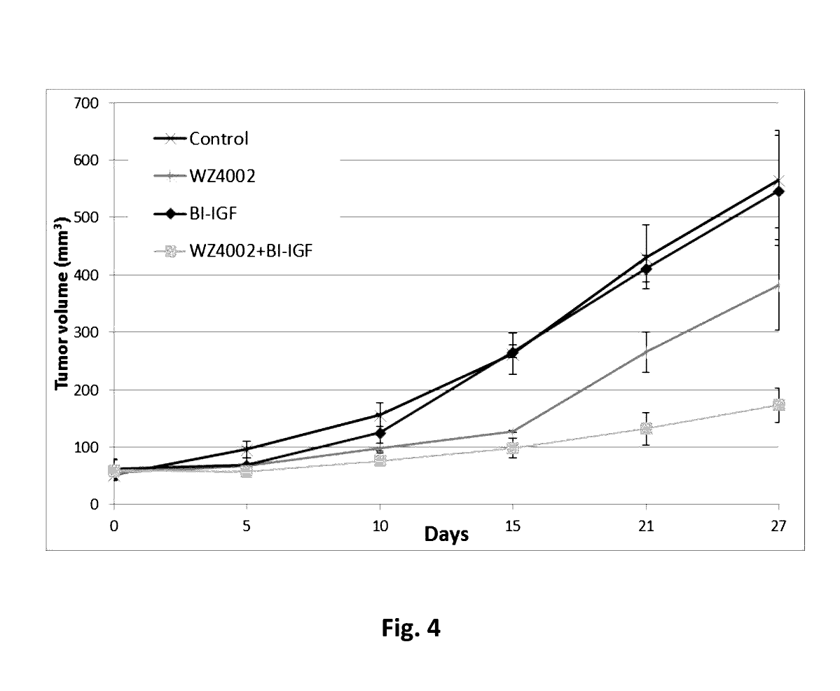

FIG. 5 shows the effect of treatment with WZ4002 and BI-IGF, alone and in combination, on the phosphorylation status of EGFR, IGF1R, and downstream signaling molecules, and on levels of apoptotic markers, in PC-9/GR/WR tumour xenografts in immunodeficient mice.

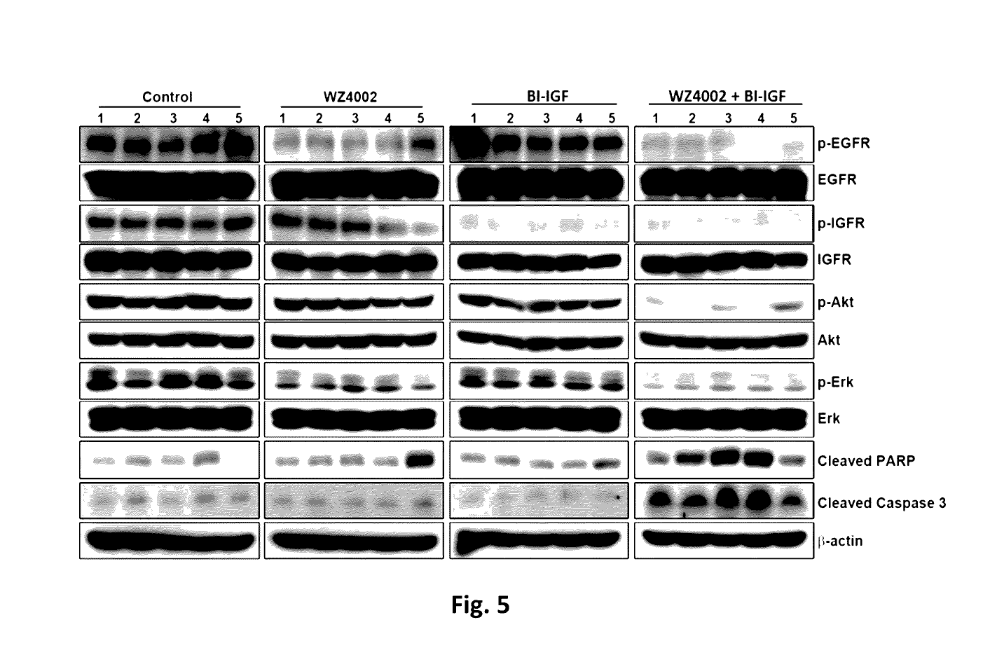

FIG. 6 shows the in vivo anti-tumor effect of Compound 1 (=HM61713) and BI-IGF, alone and in combination, on the growth of PC-9/GR/WR tumour xenografts in immunodeficient mice.

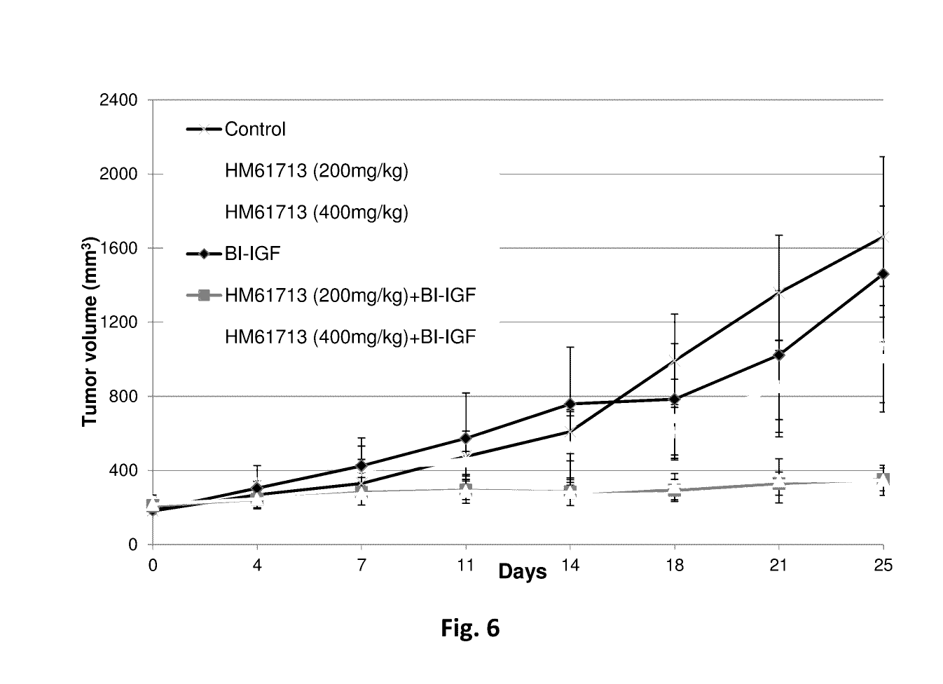

FIG. 7 shows images of individual PC-9/GR/WR tumours, after treatment with Compound 1 (=HM61713) and BI-IGF, alone and in combination.

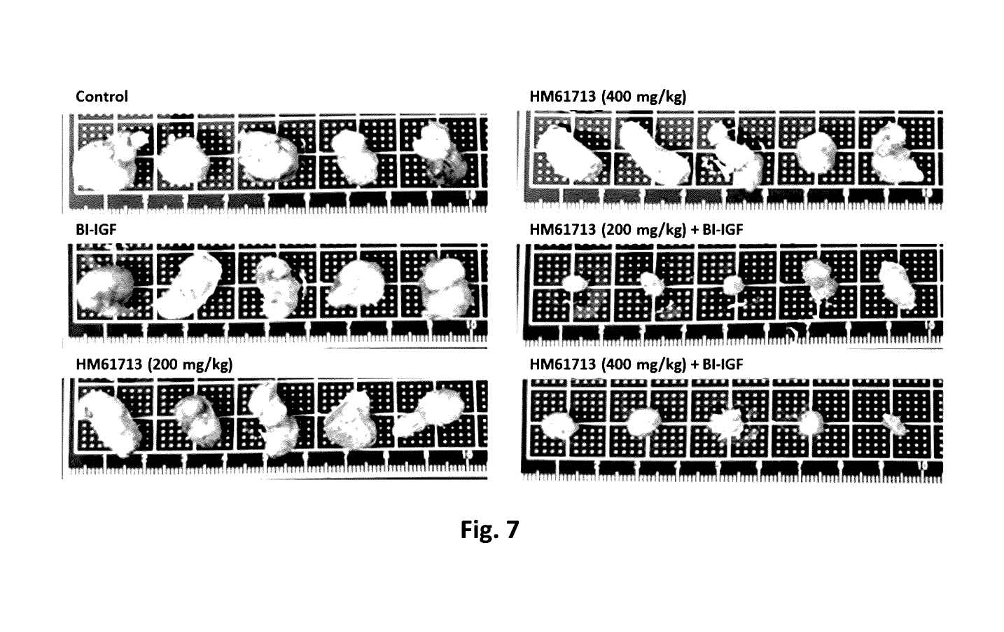

FIG. 8 shows the body weights of PC-9/GR/WR tumour-bearing immunodeficient mice treated with Compound 1 (=HM61713) and BI-IGF, alone and in combination.

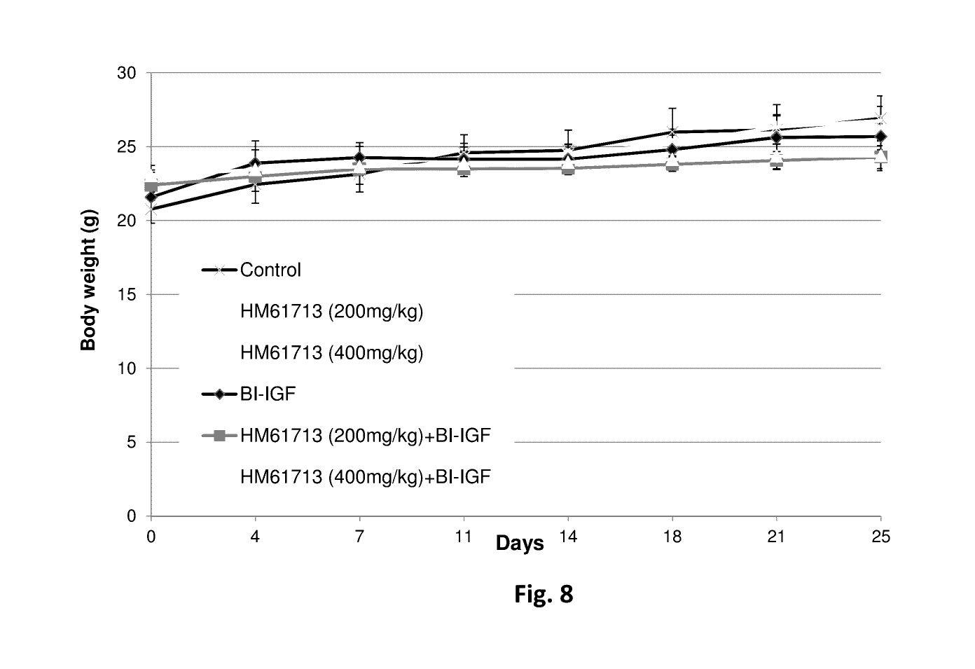

### DETAILED DESCRIPTION OF THE INVENTION

### 3G-EGFR Inhibitor

The 3G-EGFR inhibitor within the meaning of this invention is a compound which selectively inhibits EGFR mutant isoforms while sparing to some extent wild type EGFR. Preferably, this inhibition is irreversible.

Preferably, the 3G-EGFR inhibitor within this invention is selected from the group consisting of the following compounds 1 and 2 (optionally, compounds 1 and 2 are in the form of a tautomer, a pharmaceutically acceptable salt, a hydrate or a solvate; included are also all the crystalline forms of all mentioned forms).

Compound 1 (also known as HM61713): N-(3-{2-[4-(4-methyl-piperazin-1-yl)-phenylamino]-thieno[3,2-d]pyrimidin-4-yloxy}-phenyl)-acrylamide

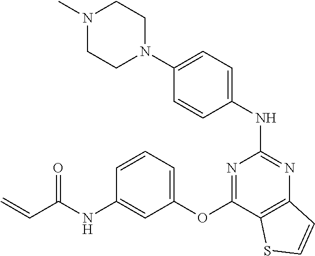 [SMILES: C=CC(=O)Nc1cccc(Oc2nc(Nc3ccc(N4CCN(C)CC4)cc3)nc3ccsc23)c1]

Compound 2 (also known as WZ4002): N-(3-{5-Chloro-2-[2-methoxy-4-(4-methyl-piperazin-1-yl)-phenylamino]-pyrimidin-4-yloxy}-phenyl)-acrylamide

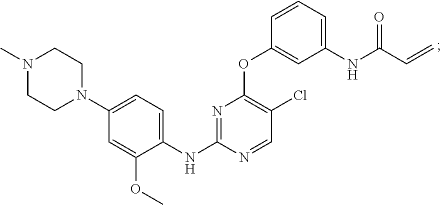 [SMILES: C=CC(=O)Nc1cccc(Oc2nc(Nc3ccc(N4CCN(C)CC4)cc3OC)ncc2Cl)c1]

Compound 1, its synthesis and properties are disclosed in WO 2011/162515 which is incorporated by reference in its entirety (example compound 1, page 33).

Compound 1 is a small molecule epidermal growth factor receptor (EGFR) mutant-specific inhibitor. It is being evaluated as a novel oral therapy for the treatment of non-small cell lung cancer (NSCLC) with EGFR mutations, including EGFR T790M (associated with acquired resistance to currently approved EGFR-targeting agents gefitinib, erlotinib, afatinib) and mutations conferring sensitivity to EGFR tyrosine-kinase inhibitors (including EGFR Deli 9, EGFR L858R etc.). In vitro data confirms that compound 1 is an irreversible EGFR mutant-specific kinase inhibitor with a more potent enzymatic inhibitory activity towards mutant forms of EGFR compared to wild type EGFR. It covalently binds to and irreversibly blocks the catalytic activity of common EGFR mutants (L858R and exon 19 deletions) and certain uncommon EGFR mutants including T790M. In cellular assays comparing EGFR mutant with EGFR wild type cell lines, compound 1 exhibits potent inhibition of proliferation of mutated cell lines at approximately 35-fold lower concentration than the one observed for inhibition of cells expressing wild type EGFR receptor. Multiple in vivo xenograft studies in mice using different NSCLC models (HCC827 (EGFRDelE746-A750) and H1975 (EGFRL858R/T790M)) confirmed the anti-tumor activity of compound 1 as a single agent. Tumor regressions were observed in all models. Anti-tumor efficacy was independent of schedule (once daily versus twice daily administration) and was tolerated by the mice at clinically relevant exposure. Compound 1 is a novel, 3^{rd} generation EGFR mutant-specific TKI, which is currently being investigated in first and second line setting for treatment of patients with EGFR-mutated NSCLC.

Compound 2, its synthesis and properties are disclosed, e.g., in WO 2010/129053 which is incorporated by reference in its entirety (example compound 2-2, page 44) and in various other prior art publications.

Additionally, the 3G-EGFR inhibitors within this invention are selected from the group consisting of osimertinib (=mereletinib=AZD9291), rociletinib (CO-1686), ASP8273, PF-06747775, avitinib (AC0010) and EGF816 and their pharmaceutically acceptable salts. Synthesis and properties of these compounds are also known in the art.

In one embodiment of the invention the 3G-EGFR inhibitor is compound 1.

In one embodiment of the invention the 3G-EGFR inhibitor is compound 2.

In a further embodiment, within the present invention it is referred to a 3G-EGFR inhibitor as described herein for use as medicament.

In a further embodiment, within the present invention it is referred to a pharmaceutical composition containing, as the active ingredient, a 3G-EGFR of the invention and as described herein.

To be used in therapy, the 3G-EGFR inhibitor, optionally in combination with one or more other active agents, is included into pharmaceutical compositions appropriate to facilitate administration to animals or humans.

Typical pharmaceutical compositions for administering the 3G-EGFR inhibitor of the invention include for example tablets, capsules, suppositories, solutions, e.g. solutions for injection (s.c., i.v., i.m.) and infusion, elixirs, emulsions or dispersible powders. The content of the pharmaceutically active compound(s) may be in the range from 0.1 to 90 wt.-%, preferably 0.5 to 50 wt.-% of the composition as a whole, e.g. in amounts which are sufficient to achieve the desired dosage range. The single dosages may, if necessary, be given several times a day to deliver the desired total daily dose.

Typical tablets may be obtained, for example, by mixing the active substance(s), optionally in combination, with known excipients, for example inert diluents such as calcium carbonate, calcium phosphate, cellulose or lactose, disintegrants such as corn starch or alginic acid or crospovidone, binders such as starch or gelatine, lubricants such as magnesium stearate or talc and/or agents for delaying release, such as carboxymethyl cellulose, cellulose acetate phthalate, or polyvinyl acetate. The tablets may be prepared by usual processes, such as e.g. by direct compression or roller compaction. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substance(s) may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing the active substance(s) may for example be prepared by mixing the active substance(s) with inert carriers such as lactose or sorbitol and packing them into gelatine capsules.

Typical suppositories may be made for example by mixing the active substance(s) with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

The 3G-EGFR inhibitors of this invention are administered by the usual methods, preferably by oral or parenteral route, most preferably by oral route. For oral administration the tablets may contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for oral use is from 1 mg to 2000 mg per day (e.g. for compound 1 the dosage in one embodiment is from 300 mg to 1200 mg per day; in a more preferred embodiment from 500 mg to 900 mg; most preferred is 800 mg per day). The dosage for intravenous use is from 1 mg to 1000 mg per hour, preferably between 5 and 500 mg per hour. All amounts given refer to the free base of compound 1 and may be proportionally higher if a pharmaceutically acceptable salt or other solid form, e.g. the dihydrochloride salt of compound 1, is used. Preferably, the daily dosage is administered once daily (q.d.).

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

Anti-IGF Antibody

An anti-IGF antibody within the meaning of this invention refers to an anti-IGF antibody molecule, which binds preferably to human IGF1 and/or IGF2.

Insulin-like growth factor-1 (IGF1; a 70 amino-acid polypeptide) and insulin-like growth factor-2 (IGF2; a 67 amino-acid polypeptide) are 7.5 kD soluble factors present in serum that can potently stimulate the growth of many mammalian cells [19]. On secretion into the bloodstream the IGFs form complexes with IGF-binding proteins (IGFBPs) which protect them from proteolytic degradation in the serum en route to their target tissues and prevents their association with the IGF receptors. IGFs are also known to be secreted in an autocrine or paracrine manner in target tissues themselves. This is known to occur during normal fetal development where the IGFs play a key role in the growth of tissues, bone and organs. It is also seen in many cancer tissues where there is thought to be paracrine signaling between tumour cells and stromal cells or autocrine IGF production by the tumour cells themselves [20].

IGF1 and IGF2 are able to bind to the IGF1 receptor (IGF1R) expressed on many normal tissues, which functionally is a 460 kD heterotetramer consisting of a dimerised alpha- and beta-subunit, with similar affinities [21]. IGF2 can also bind to the IGF2 receptor, which is thought to prevent IGF2 from binding and signaling through the IGF1R. In this respect the IGF2R has been demonstrated to be a tumour suppressor protein. The IGF1R is structurally similar to the insulin receptor which exists in two forms, IR-A and IR-B, which differ by an alternatively spliced 12 amino acid exon deletion in the extracellular domain of IR-A. IR-B is the predominant IR isoform expressed in most normal adult tissues where it acts to mediate the effects of insulin on metabolism. IR-A on the other hand is known to be highly expressed in developing fetal tissues but not in adult normal tissues. Recent studies have also shown that IR-A, but not IR-B, is highly expressed in some cancers. The exon deletion in IR-A has no impact on insulin binding but does cause a small conformational change that allows IGF2 to bind with much higher affinity than for IR-B [22, 23]. Thus, because of its expression in cancer tissues and increased propensity for IGF2 binding, IR-A may be as important as IGF1R in mediating the mitogenic effects of IGF2 in cancer.

Binding of the IGFs to IGF1R triggers a complex intracellular signaling cascade which results in activation of proteins that stimulate proliferation and survival [19].

Unlike the EGFR and Her2neu receptors there is no known amplification of the IGF1R or IR-A receptors in cancers indicating that receptor activation is controlled by the presence of active ligand. There is a very large body of scientific, epidemiological and clinical literature implicating a role for the IGFs in the development, progression and metastasis of many different cancer types [24, 19].

By blocking receptor-ligand binding, ligand-induced receptor signaling through the tyrosine kinase activity of the receptor is reduced or prevented. Such antibodies are generally referred to as neutralizing antibodies.

The term “antibody” encompasses antibodies, antibody fragments, antibody-like molecules and conjugates with any of the above. Antibodies include, but are not limited to, poly- or monoclonal, chimeric, humanized, human, mono-, bi- or multispecific antibodies.

The term “antibody” shall encompass complete immunoglobulins as they are produced by lymphocytes and for example present in blood sera, monoclonal antibodies secreted by hybridoma cell lines, polypeptides produced by recombinant expression in host cells, which have the binding specificity of immunoglobulins or monoclonal antibodies, and molecules which have been derived from such immunoglobulins, monoclonal antibodies, or polypeptides by further processing while retaining their binding specificity. In particular, the term “antibody” includes complete immunoglobulins comprising two heavy chains and two light chains. In another embodiment, the term encompasses a fragment of an immunoglobulin, like Fab fragments. In another embodiment, the term “antibody” encompasses a polypeptide having one or more variable domains derived from an immunobulin, like single chain antibodies (scFv), single domain antibodies, and the like.

Preferably, the anti-IGF antibody within this invention is a human anti-IGF antibody.

In another embodiment, the anti-IGF antibody within this invention refers to an anti-IGF antibody molecule having heavy chain complementary determining regions of SEQ ID NO: 1 (HCDR1), SEQ ID NO: 2 (HCDR2), and SEQ ID NO: 3 (HCDR3) and light chain determining regions of SEQ ID NO: 4 (LCDR1), SEQ ID NO: 5 (LCDR2), and SEQ ID NO: 6 (LCDR3).

In another embodiment, the anti-IGF antibody within this invention refers to an anti-IGF antibody molecule having a heavy chain variable region of SEQ ID NO: 7 and a light chain variable region of SEQ ID NO: 8.

In another embodiment, the anti-IGF antibody within this invention refers to an anti-IGF antibody molecule having a heavy chain of SEQ ID NO: 9, and a light chain of SEQ ID NO: 10 (referred to herein as “BI-IGF”).

Manufacture and therapeutic use of the aforementioned antibody is disclosed in WO 2010/066868, WO 2013/060872 and WO 2014/135611. In particular, these documents provide a sufficient disclosure of the method of preparing the antibody molecule used in the present invention.

In another embodiment, the anti-IGF antibody within this invention refers to the anti-IGF antibody molecule MEDI-573 (=dusigitumab) as known in the art.

In a further embodiment, within the present invention it is referred to an antibody molecule as described herein for use as medicament.

In a further embodiment, within the present invention it is referred to a pharmaceutical composition containing, as the active ingredient, an anti-IGF antibody molecule, preferably a full antibody, of the invention as described herein.

To be used in therapy, the anti-IGF antibody molecule, optionally in combination with one or more other active agents, is included into pharmaceutical compositions appropriate to facilitate administration to animals or humans.

Typical formulations of the anti-IGF antibody molecule can be prepared by mixing the anti-IGF antibody molecule with physiologically acceptable carriers, excipients or stabilizers, in the form of lyophilized or otherwise dried formulations or aqueous solutions or aqueous or non-aqueous suspensions. Carriers, excipients, modifiers or stabilizers are nontoxic at the dosages and concentrations employed. They include buffer systems such as phosphate, citrate, acetate and other inorganic or organic acids and their salts; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone or polyethylene glycol (PEG); amino acids such as glycine, glutamine, asparagine, histidine, arginine, or lysine; monosaccharides, disaccharides, oligosaccharides or polysaccharides and other carbohydrates including glucose, mannose, sucrose, trehalose, dextrins or dextrans; chelating agents such as EDTA; sugar alcohols such as, mannitol or sorbitol; salt-forming counter-ions such as sodium; metal complexes (e.g., Zn-protein complexes); and/or ionic or non-ionic surfactants such as TWEEN™ (polysorbates), PLURONICS™ or fatty acid esters, fatty acid ethers or sugar esters. Also organic solvents can be contained in the antibody formulation such as ethanol or isopropanol. The excipients may also have a release-modifying or absorption-modifying function.

The anti-IGF antibody molecules may also be dried (freeze-dried, spray-dried, spray-freeze dried, dried by near or supercritical gases, vacuum dried, air-dried), precipitated or crystallized or entrapped in microcapsules that are prepared, for example, by coacervation techniques or by interfacial polymerization using, for example, hydroxymethylcellulose or gelatin and poly-(methylmethacylate), respectively, in colloidal drug delivery systems (for example, liposomes, albumin microspheres, microemulsions, nano-particles and nanocapsules), in macroemulsions or precipitated or immobilized onto carriers or surfaces, for example by pcmc technology (protein coated microcrystals). Such techniques are disclosed in Remington, 2005.

Naturally, the formulations to be used for in vivo administration must be sterile; sterilization may be accomplished be conventional techniques, e.g. by filtration through sterile filtration membranes.

In another preferred embodiment the antibody is formulated in an aqueous buffer composition for parenteral (intravenous) infusion or injection at an antibody concentration of 10 mg/mL, said buffer comprising 24.2 mM histidine, 3.88% mannitol, 0.97% sucrose, 0.02% polysorbate 20, pH 6.0. For intravenous infusion, the pharmaceutical composition may be diluted with a physiological solution, e.g. with 0.9% sodium chloride or G5 solution.

The antibody may be administered to the patient at a dose between 1 mg/kg to 20 mg/kg, by one or more separate administrations, or by continuous infusion, e.g. infusion over 1 hour. A typical treatment schedule usually involves administration of the antibody once every week to once every three weeks.

For example, a weekly dose could be 5, 10, or 15 mg/kg. Preferably, the antibody is prepared at a concentration of 10 mg/mL of antibody. The antibody may preferably be administered to a patient as a 750 mg (up to 1000 mg) total dose by one hour i.v. infusion, to be repeated once a week until disease progression

Combination Therapy

Within this invention it is to be understood that the combinations, compositions, kits, methods, uses or compounds for use according to this invention may envisage the simultaneous, concurrent, sequential, successive, alternate or separate administration of the active ingredients or components. It will be appreciated that the 3G-EGFR inhibitor and the anti-IGF antibody can be administered formulated either dependently or independently, such as e.g. the 3G-EGFR inhibitor and the anti-IGF antibody may be administered either as part of the same pharmaceutical composition/dosage form or, preferably, in separate pharmaceutical compositions/dosage forms.

In this context, “combination” or “combined” within the meaning of this invention includes, without being limited, fixed and non-fixed (e.g. free) forms (including kits) and uses, such as e.g. the simultaneous, concurrent, sequential, successive, alternate or separate use of the components or ingredients.

The administration of the 3G-EGFR inhibitor and the anti-IGF antibody may take place by co-administering the active components or ingredients, such as e.g. by administering them simultaneously or concurrently in one single or in two separate formulations or dosage forms. Alternatively, the administration of the 3G-EGFR inhibitor and the anti-IGF antibody may take place by administering the active components or ingredients sequentially or in alternation, such as e.g. in two separate formulations or dosage forms.

For example, simultaneous administration includes administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent during a second time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

The elements of the combinations of this invention may be administered (whether dependently or independently) by methods customary to the skilled person, e.g. by oral, enterical, parenteral (e.g., intramuscular, intraperitoneal, intravenous, transdermal or subcutaneous injection, or implant), nasal, vaginal, rectal, or topical routes of administration and may be formulated, alone or together, in suitable dosage unit formulations containing conventional non-toxic pharmaceutically acceptable carriers, excipients and/or vehicles appropriate for each route of administration.

Accordingly, in one aspect of the invention, the invention provides a method of treating and/or preventing an oncological or hyperproliferative disease, in particular cancer (such as e.g. the cancer disorders described herein), comprising administering to a patient in need thereof a therapeutically effective amount of a 3G-EGFR inhibitor and an anti-IGF antibody (each as described herein), simultaneously, concurrently, sequentially, successively, alternately or separately.

In another aspect, the invention provides a 3G-EGFR inhibitor as described herein for use in a method of treating and/or preventing an oncological or hyperproliferative disease, in particular cancer, said method comprising administering the 3G-EGFR inhibitor simultaneously, concurrently, sequentially, successively, alternately or separately with an anti-IGF antibody as described herein.

In another aspect, the invention provides an anti-IGF antibody as described herein for use in a method of treating and/or preventing an oncological or hyperproliferative disease, in particular cancer, said method comprising administering the anti-IGF antibody simultaneously, concurrently, sequentially, successively, alternately or separately with a 3G-EGFR inhibitor as described herein.

In another aspect, the invention provides the use of a 3G-EGFR inhibitor and/or an anti-IGF antibody, each as described herein, for preparing a pharmaceutical composition for treating and/or preventing an oncological or hyperproliferative disease, in particular cancer (such as e.g. a cancer disease as described herein), in combination.

In another aspect, the invention provides a combination, composition or kit comprising, consisting of, or consisting essentially of a 3G-EGFR inhibitor and an anti-IGF antibody, each as described herein, and optionally one or more pharmaceutically acceptable carriers, excipients and/or vehicles, e.g. for simultaneous, concurrent, sequential, successive, alternate or separate use of the active components in therapy.

In a preferred embodiment, the 3G-EGFR inhibitor is to be administered orally.

In another preferred embodiment, the anti-IGF antibody is to be administered parenterally, by infusion or injection.

The “therapeutically effective amount” of the active compound(s) to be administered is the minimum amount necessary to prevent, ameliorate, or treat a disease or disorder.

The combinations of this invention may be administered at therapeutically effective single or divided daily doses. The active components of the combination may be administered in such doses which are therapeutically effective in monotherapy, or in such doses which are lower than the doses used in monotherapy, but when combined result in a desired therapeutically effective amount.

In particular embodiments of this invention, the combinations, compositions, kits, methods, uses and compounds for use according to this invention relate to such combinations, compositions, kits, methods, uses and compounds for use in which the 3G-EGFR inhibitor is selected from the group consisting of the compounds 1 and 2 indicated herein above and the anti-IGF antibody is an anti-IGF antibody molecule having heavy chain CDRs comprising the amino acid sequences of SEQ ID NO:1 (CDR1), SEQ ID NO:2 (CDR2) and SEQ ID NO:3 (CDR3) and light chain CDRs comprising the amino acid sequences of SEQ ID NO:4 (CDR1), SEQ ID NO:5 (CDR2) and SEQ ID NO:6 (CDR3).

In more particular embodiments of this invention, the combinations, compositions, kits, methods, uses and compounds for use according to this invention relate to such combinations, compositions, kits, methods, uses and compounds for use in which the 3G-EGFR inhibitor is selected from the group consisting of the compounds 1 and 2 indicated herein above and the anti-IGF antibody is an anti-IGF antibody molecule having a variable heavy chain comprising the amino acid sequence of SEQ ID NO:7 and a variable light chain comprising the amino acid sequence of SEQ ID NO:8.

In certain preferred embodiments of this invention, the combinations, compositions, kits, methods, uses and compounds for use according to this invention relate to such combinations, compositions, kits, methods, uses and compounds for use in which the 3G-EGFR inhibitor is selected from the group consisting of the compounds 1 and 2 indicated herein above and the anti-IGF antibody is an anti-IGF antibody molecule having a heavy chain comprising the amino acid sequence of SEQ ID NO:9 and a light chain comprising the amino acid sequence of SEQ ID NO:10.

In certain embodiments (embodiments A) of this invention, the combinations, compositions, kits, methods, uses and compounds for use according to this invention refer to such individual pairs of the 3G-EGFR inhibitor and the anti-IGF antibody according to the embodiment entries A1 to A16 (table i):

TABLE i

| Embodi- | | |
|---|---|---|
| ment | 3G-EGFR inhibitor | anti-IGF antibody |
| A1 | Compound 1 (=HM61713) | Antibody designated as BI-IGF |
| A2 | Compound 2 (=WZ4002) | Antibody designated as BI-IGF |
| A3 | Osimertinib | Antibody designated as BI-IGF |
| | (=Mereletinib = AZD9291) | |
| A4 | Rociletinib (=CO-1686) | Antibody designated as BI-IGF |
| A5 | Compound 1 (=HM61713) | MEDI-573 (=dusigitumab) |
| A6 | Compound 2 (=WZ4002) | MEDI-573 (=dusigitumab) |
| A7 | Osimertinib | MEDI-573 (=dusigitumab) |
| | (=Mereletinib = AZD9291) | |
| A8 | Rociletinib (=CO-1686) | MEDI-573 (=dusigitumab) |
| A9 | EGF816 | Antibody designated as BI-IGF |
| A10 | EGF816 | MEDI-573 (=dusigitumab) |
| A11 | ASP8273 | Antibody designated as BI-IGF |
| A12 | ASP8273 | MEDI-573 (=dusigitumab) |
| A13 | PF-06747775 | Antibody designated as BI-IGF |
| A14 | PF-06747775 | MEDI-573 (=dusigitumab) |
| A15 | Avitinib (AC0010) | Antibody designated as BI-IGF |
| A16 | Avitinib (AC0010) | MEDI-573 (=dusigitumab) |

The combinations, compositions, kits, uses, methods and compounds for use according to the present invention are useful for the treatment and/or prevention of oncological and hyperproliferative disorders.

In certain embodiments the combinations, compositions, kits, uses, methods and compounds for use according to the present invention are useful for the treatment of oncological and hyperproliferative disorders.

In certain embodiments, the hyperproliferative disorder is cancer.

Cancers are classified in two ways: by the type of tissue in which the cancer originates (histological type) and by primary site, or the location in the body, where the cancer first developed. The most common sites in which cancer develops include the skin, lung, breast, prostate, colon and rectum, cervix and uterus as well as the hematological compartment.

The combinations, compositions, kits, uses, methods and compounds for use according to the invention are useful in the treatment of a variety of cancer diseases, including, for example, but not limited to the following:
brain related cancer such as adult brain tumour, childhood brain stem glioma, childhood cerebellar astrocytoma, childhood cerebral astrocytoma/malignant glioma, childhood ependymoma, childhood medulloblastoma, childhood supratentorial primitive neuroectodermal tumours, childhood visual pathway and hypothalamic glioma and other childhood brain tumours;
breast cancer;
digestive/gastrointestinal related cancer such as anal cancer, extrahepatic bile duct cancer, gastrointestinal carcinoid tumour, cholangiocarcinoma, colon cancer, esophageal cancer, gallbladder cancer, adult primary liver cancer (hepatocellular carcinoma, hepatoblastoma) childhood liver cancer, pancreatic cancer, rectal cancer, small intestine cancer and stomach (gastric) cancer;
endocrine related cancer such as adrenocortical carcinoma, gastrointestinal carcinoid tumour, islet cell carcinoma (endocrine pancreas), parathyroid cancer, pheochromocytoma, pituitary tumour and thyroid cancer;
eye related cancer such as intraocular melanoma, and retinoblastoma;
genitourinary related cancer such as bladder cancer, kidney (renal cell) cancer, penile cancer, prostate cancer, transitional cell renal pelvis and ureter cancer, testicular cancer, urethral cancer, Wilms' tumour and other childhood kidney tumours;
germ cell related cancer such as childhood extracranial germ cell tumour, extragonadal germ cell tumour, ovarian germ cell tumour and testicular cancer;
gynecologic cancer such as cervical cancer, endometrial cancer, gestational trophoblastic tumour, ovarian epithelial cancer, ovarian germ cell tumour, ovarian low malignant potential tumour, uterine sarcoma, vaginal cancer and vulvar cancer;
head and neck related cancer such as hypopharyngeal cancer, laryngeal cancer, lip and oral cavity cancer, metastatic squamous neck cancer with occult primary, nasopharyngeal cancer, oropharyngeal cancer, paranasal sinus and nasal cavity cancer (e.g. sinonasal squamouns cell carcinoma), parathyroid cancer and salivary gland cancer;
hematologic/blood related cancer such as leukemias, such as adult acute lymphoblastic leukemia, childhood acute lymphoblastic leukemia, adult acute myeloid leukemia, childhood acute myeloid leukemia, chronic lymphocytic leukemia, chronic myelogenous leukemia and hairy cell leukemia; and lymphomas, such as AIDS-related lymphoma, cutaneous T-cell lymphoma, adult Hodgkin's lymphoma, childhood Hodgkin's lymphoma, Hodgkin's lymphoma during pregnancy, mycosis fungoides, adult non-Hodgkin's lymphoma, childhood non-Hodgkin's lymphoma, non-Hodgkin's lymphoma during pregnancy, primary central nervous system lymphoma, Sezary syndrome, cutaneous T-cell lymphoma and Waldenström's macroglobulinemia and other hematologic/blood related cancer such as chronic myeloproliferative disorders, multiple myeloma/plasma cell neoplasm, myelodysplastic syndromes and myelodysplastic/myeloproliferative diseases;
musculoskeletal related cancer such as Ewing's family of tumours, osteosarcoma, malignant fibrous histiocytoma of bone, childhood rhabdomyosarcoma, adult soft tissue sarcoma, childhood soft tissue sarcoma and uterine sarcoma; hemangiosarcomas and angiosarcoma;
neurologic related cancer such as adult brain tumour, childhood brain tumour, brain stem glioma, cerebellar astrocytoma, cerebral astrocytoma/malignant glioma, ependmoma, medulloblastoma, supratentorial primitive neuroectodermal tumours, visual pathway and hypothalamic glioma and other brain tumours such as neuroblastoma, pituitary tumour and primary central nervous system lymphoma;
respiratory/thoracic related cancer such as non-small cell lung cancer (NSCLC), small cell lung cancer (SCLC), squamous cell carcinoma (SCC) of the lung, malignant mesothelioma, thymoma and thymic carcinoma;
skin related cancer such as cutaneous T-cell lymphoma, Kaposi's sarcoma, melanoma, Merkel cell carcinoma and skin cancer;
small blue round cell tumours.

In a further embodiment, the combinations, compositions, kits, uses, methods and compounds for use of the invention are beneficial in the treatment of cancers of the hematopoietic system including leukemias, lymphomas and myelomas, cancers of the gastrointestinal tract including esophageal, gastric, colorectal, pancreatic, liver and gall bladder and bile duct cancer; kidney, prostate and bladder cancer; gynecological cancers including breast, ovarian, cervical and endometrial cancer; skin and head and neck cancers including malignant melanomas; pediatric cancers like Wilms' tumour, neuroblastoma and Ewing′sarcoma; brain cancers like glioblastoma; sarcomas like osteosarcoma, soft tissue sarcoma, rhabdomyosarcoma, hemangiosarcoma; lung cancer including non-small cell lung cancer, mesothelioma and thyroid cancer.

In a further embodiment of the invention, the combinations, compositions, kits, uses, methods and compounds for use according to the invention are used to treat a cancer selected from the group consisting of non-small cell lung cancer (NSCLC) (including for example locally advanced or metastatic NSCLC (stage IIIB/IV)).

In a further embodiment of the invention, the combinations, compositions, kits, uses, methods and compounds for use according to the invention are used to treat non-small cell lung cancer (NSCLC) (including for example locally advanced or metastatic NSCLC (stage IIIB/IV), NSCLC adenocarcinoma, NSCLC with squamous histology, NSCLC with non-squamous histology).

In a further embodiment of the invention, the combinations, compositions, kits, uses, methods and compounds for use according to the invention are used in the treatment of non-small cell lung cancer (NSCLC), in particular NSCLC adenocarcinoma.

In a further embodiment of the invention, the combinations, compositions, kits, uses, methods and compounds for use according to the invention are used in the treatment of non-small cell lung cancer (NSCLC) characterized by aberrant activation, or amplification, or mutations of EGFR.

In a further embodiment of the invention, the combinations, compositions, kits, uses, methods and compounds for use according to the invention are used in the treatment of a cancer harboring one or more EGFR mutation.

In a further embodiment of the invention, the combinations, compositions, kits, uses, methods and compounds for use according to the invention are used in the treatment of a cancer harboring an EGFR exon 20 insertion, an EGFR exon 19 deletion (Del19), an EGFR L858R mutation, an EGFR T790M mutation, or any combination thereof.

In a further embodiment of the invention, the combinations, compositions, kits, uses, methods and compounds for use according to the invention are used in the treatment of a cancer harboring one or more EGFR mutations wherein at least one EGFR mutation is selected from Del19 (deletion in exon 19), L858R and T790M.

In a further embodiment of the invention, the combinations, compositions, kits, uses, methods and compounds for use according to the invention are used in the treatment of a cancer harboring the EGFR mutation Del19.

In a further embodiment of the invention, the combinations, compositions, kits, uses, methods and compounds for use according to the invention are used in the treatment of a cancer harboring the EGFR mutation L858R.

In a further embodiment of the invention, the combinations, compositions, kits, uses, methods and compounds for use according to the invention are used in the treatment of a cancer harboring the EGFR mutation T790M.

In a further embodiment of the invention, the combinations, compositions, kits, uses, methods and compounds for use according to the invention are used in the treatment of a cancer harboring at least two EGFR mutations selected from the group consisting of Del19/T790M and L858R/T790M.

In a further embodiment of the invention, the combinations, compositions, kits, uses, methods and compounds for use according to the invention are used in the treatment of non-small cell lung cancer (NSCLC), in particular NSCLC adenocarcinoma, harboring an EGFR exon 20 insertion, an EGFR exon 19 deletion (Del19), an EGFR L858R mutation, an EGFR T790M mutation, or any combination thereof.

In a further embodiment of the invention, the combinations, compositions, kits, uses, methods and compounds for use according to the invention are used in the treatment of non-small cell lung cancer (NSCLC), in particular NSCLC adenocarcinoma, harboring one or more EGFR mutations wherein at least one EGFR mutation is selected from Del19 (deletion in exon 19), L858R and T790M.

In a further embodiment of the invention, the combinations, compositions, kits, uses, methods and compounds for use according to the invention are used in the treatment of non-small cell lung cancer (NSCLC), in particular NSCLC adenocarcinoma, harboring at least two EGFR mutations selected from the group consisting of Del19/T790M and L858R/T790M.

In a further embodiment of the invention, the combinations, compositions, kits, uses, methods and compounds for use according to the invention are used in the treatment of non-small cell lung cancer (NSCLC), in particular NSCLC adenocarcinoma, harboring the EGFR mutation Deli 9.

In a further embodiment of the invention, the combinations, compositions, kits, uses, methods and compounds for use according to the invention are used in the treatment of non-small cell lung cancer (NSCLC), in particular NSCLC adenocarcinoma, harboring the EGFR mutation L858R.

In a further embodiment of the invention, the combinations, compositions, kits, uses, methods and compounds for use according to the invention are used in the treatment of non-small cell lung cancer (NSCLC), in particular NSCLC adenocarcinoma, harboring the EGFR mutation T790M.

The therapeutic applicability of the combination therapy according to this invention may include first line, second line, third line or further lines of treatment of patients. The cancer may be metastatic, recurrent, relapsed, resistant or refractory to one or more anti-cancer treatments. Thus, the patients may be treatment naïve, or may have received one or more previous anti-cancer therapies, which have not completely cured the disease.

Patients with relapse and/or with resistance to one or more anti-cancer agents (e.g. the single components of the combination, or standard chemotherapeutics) are also amenable for combined treatment according to this invention, e.g. for second or third line treatment cycles (optionally in further combination with one or more other anti-cancer agents), e.g. as add-on combination or as replacement treatment.

Accordingly, some of the disclosed combination therapies of this invention are effective at treating subjects whose cancer has relapsed, or whose cancer has become drug resistant or multi-drug resistant, or whose cancer has failed one, two or more lines of mono- or combination therapy with one or more anti-cancer agents (e.g. the single components of the combination, or standard chemotherapeutics).

A cancer which initially responded to an anti-cancer drug can relapse and it becomes resistant to the anti-cancer drug when the anti-cancer drug is no longer effective in treating the subject with the cancer, e.g. despite the administration of increased dosages of the anti-cancer drug. Cancers that have developed resistance to two or more anti-cancer drugs are said to be multi-drug resistant.

Accordingly, in some methods of combination treatment of this invention, treatment with a combination according to this invention administered secondly or thirdly is begun if the patient has resistance or develops resistance to one or more agents administered initially or previously. The patient may receive only a single course of treatment with each agent or multiple courses with one, two or more agents.

In certain instances, combination therapy according to this invention may hence include initial or add-on combination, replacement or maintenance treatment.

In a further embodiment of the invention, the combinations, compositions, kits, uses, methods and compounds for use according to the invention are used in the treatment of cancers/cancer patients (suffering from cancers as described herein, in particular suffering from NSCLC as described herein) which are treatment naïve, i.e. their cancer disease has not been treated previously. In further embodiments the cancers/cancer patients (suffering from cancers as described herein, in particular suffering from NSCLC as described herein) have been previously treated with 1^{st} generation EGFR TKIs selected from erlotinib and gefitinib. In further embodiments the cancers/cancer patients (suffering from cancers as described herein, in particular suffering from NSCLC as described herein) have been previously treated with 2^{nd} generation EGFR TKIs selected from afatinib and dacomitinib.

The present invention is not to be limited in scope by the specific embodiments described herein. Various modifications of the invention in addition to those described herein may become apparent to those skilled in the art from the present disclosure. Such modifications are intended to fall within the scope of the appended claims.

All patent applications cited herein are hereby incorporated by reference in their entireties.

Further embodiments, features and advantages of the present invention may become apparent from the following examples. The following examples serve to illustrate, by way of example, the principles of the invention without restricting it.

### REFERENCE

1. Mok T S, Wu Y L, Thongprasert S et al. Gefitinib or carboplatin-paclitaxel in pulmonary adenocarcinoma. N Engl J Med 2009; 361: 947-957.
2. Zhou C, Wu Y L, Chen G et al. Erlotinib versus chemotherapy as first-line treatment for patients with advanced EGFR mutation-positive non-small-cell lung cancer (OPTIMAL, CTONG-0802): a multicentre, open-label, randomised, phase 3 study. Lancet Oncol 2011; 12: 735-742.
3. Paez J G, Janne P A, Lee J C et al. EGFR mutations in lung cancer: correlation with clinical response to gefitinib therapy. Science 2004; 304: 1497-1500.
4. Lynch T J, Bell D W, Sordella R et al. Activating mutations in the epidermal growth factor receptor underlying responsiveness of non-small-cell lung cancer to gefitinib. N Engl J Med 2004; 350: 2129-2139.
5. Pao W, Miller V, Zakowski M et al. EGF receptor gene mutations are common in lung cancers from “never smokers” and are associated with sensitivity of tumors to gefitinib and erlotinib. Proc Natl Acad Sci USA 2004; 101: 13306-13311.
6. Rosell R, Carcereny E, Gervais R et al. Erlotinib versus standard chemotherapy as first-line treatment for European patients with advanced EGFR mutation-positive non-small-cell lung cancer (EURTAC): a multicentre, open-label, randomised phase 3 trial. Lancet Oncol 2012; 13: 239-246.
7. Fukuoka M, Wu Y L, Thongprasert S et al. Biomarker analyses and final overall survival results from a phase III, randomized, open-label, first-line study of gefitinib versus carboplatin/paclitaxel in clinically selected patients with advanced non-small-cell lung cancer in Asia (I PASS). J Clin Oncol 2011; 29: 2866-2874.
8. Kobayashi S, Boggon T J, Dayaram T et al. EGFR mutation and resistance of non-small-cell lung cancer to gefitinib. N Engl J Med 2005; 352: 786-792.
9. Morita S, Okamoto I, Kobayashi K et al. Combined survival analysis of prospective clinical trials of gefitinib for non-small cell lung cancer with EGFR mutations. Clin Cancer Res 2009; 15: 4493-4498.
10. Pao W, Miller V A, Politi K A et al. Acquired resistance of lung adenocarcinomas to gefitinib or erlotinib is associated with a second mutation in the EGFR kinase domain. PLoS Med 2005; 2: e73.
11. Riely G J, Pao W, Pham D et al. Clinical course of patients with non-small cell lung cancer and epidermal growth factor receptor exon 19 and exon 21 mutations treated with gefitinib or erlotinib. Clin Cancer Res 2006; 12: 839-844.
12. Zhou W, Ercan D, Chen L et al. Novel mutant-selective EGFR kinase inhibitors against EGFR T790M. Nature 2009; 462: 1070-1074.
13. Cross D A, Ashton S E, Ghiorghiu S et al. AZD9291, an irreversible EGFR TKI, overcomes T790M-mediated resistance to EGFR inhibitors in lung cancer. Cancer Discov 2014; 4: 1046-1061.
14. Planchard D. O10.4AZD9291 a novel EGFR-TKI that overcomes T790M-mediated resistance in NSCLC. Ann Oncol 2015; 26 Suppl 2: ii14.
15. Soria J C, Sequist L V, Goldman J et al. O10.3Rociletinib: an oral, irreversible, highly selective small molecule inhibitor of mutant EGFR including T790M. Ann Oncol 2015; 26 Suppl 2: ii14.
16. Steuer C E, Khuri F R, Ramalingam S S. The next generation of epidermal growth factor receptor tyrosine kinase inhibitors in the treatment of lung cancer. Cancer 2015; 121: E1-6.
17. Jiang T, Zhou C. Clinical activity of the mutant-selective EGFR inhibitor AZD9291 in patients with EGFR inhibitor-resistant non-small cell lung cancer. Transl Lung Cancer Res 2014; 3: 370-372.
18. Finlay M R, Anderton M, Ashton S et al. Discovery of a potent and selective EGFR inhibitor (AZD9291) of both sensitizing and T790M resistance mutations that spares the wild type form of the receptor. J Med Chem 2014; 57: 8249-8267.
19. Pollak M N et al, 2004. Insulin-like growth factors and neoplasia. Nature 4:505-518.
20. LeRoith D et al, 2003. The insulin-like growth factor system and cancer. Cancer Lett 195(2):127-37.
21. Rubin R et al, 1995. Insulin-like growth factor-I receptor. Its role in cell proliferation, apoptosis, and tumorigenicity. Lab Invest 73(3):311-31.
22. Frasca F et al, 1999. Insulin receptor isoform A, a newly recognized, high-affinity insulin-like growth factor II receptor in fetal and cancer cells. Mol Cell Biol 19(5):3278-88.
23. Pandini G et al, 2002. Insulin/insulin-like growth factor I hybrid receptors have different biological characteristics depending on the insulin receptor isoform involved. J Biol Chem 277(42):39684-95.
24. Jerome L et al, 2003. Deregulation of the IGF axis in cancer: epidemiological evidence and potential therapeutic interventions. Endocr Relat Cancer 10(4):561-78.
25. Cortot A B et al, 2013. Resistance to irreversible EGF receptor tyrosine kinase inhibitors through a multistep mechanism involving the IGF1R pathway. Cancer Res 73 (2):834-843.

### EXAMPLES

### Cell Culture and Reagents

The human NSCLC cell line PC-9 is commercially available at RIKEN BRC, Cat #RCB4455. Cells were cultured in RPMI 1640 medium containing 10% FBS, 2 mmol/L L-glutamine, and 100 units/mL of penicillin and streptomycin, and maintained at 37° C. in a humidified chamber containing 5% CO₂. WZ4002 (compound 2) was purchased from Selleck Chemicals (Houston, Tex.). BI-IGF was obtained according to methods described in WO 2010/066868.

Generation of WZ-4002-Resistant Cells

PC-9/GR(L) cells were established as part of a previous study (Mok T S, Wu Y L, Thongprasert S et al. Gefitinib or carboplatin-paclitaxel in pulmonary adenocarcinoma. N Engl J Med 2009; 361: 947-957). These cells were additionally cultured under the continuous stress of gefitinib or erlotinib treatment for 6 months. This resistant subline was designated PC-9/GR. To create a WZ4002-resistant cell lines, PC-9/GR cells were exposed to increasing concentrations of WZ4002 similar to descriptions in previous studies (Rho J K, Choi Y J, Kim S Y et al. MET and AXL inhibitor NPS-1034 exerts efficacy against lung cancer cells resistant to EGFR kinase inhibitors because of MET or AXL activation. Cancer Res 2014; 74: 253-262; Rho J K, Choi Y J, Lee J K et al. The role of MET activation in determining the sensitivity to epidermal growth factor receptor tyrosine kinase inhibitors. Mol Cancer Res 2009; 7: 1736-1743). WZ4002-resistant cells are referred to as PC-9/GR/WR cells.

Proliferation Assay (MTT Assay)

Cells (5×10³) were seeded in 96-well sterile plastic plates overnight and then treated with increasing concentrations of WZ4002, alone or in combination with a fixed concentration of BI-IGF (100 μg/mL). After 72 h, 15 μL of MTT solution (5 mg/mL) was added to each well and plates were incubated for 4 h. Crystalline formazan was solubilized with 100 μL of a 10% (w/v) SDS solution for 24 h, and then absorbance at 595 nm was read spectrophotometrically using a microplate reader. The results are representative of at least three, independent experiments, and the error bars signify standard deviations (SDs).

Western Blot Analysis

PC-9/GR/WR cells were treated with WZ4002 (1 μM) and BI-IGF (100 μg/mL), alone and in combination. After 48 h, cells were harvested and lysed in buffer containing 137 mmol/L NaCl, 15 mmol/L EGTA, 0.1 mmol/L sodium orthovanadate, 15 mmol/L MgCl₂, 0.1% Triton X-100, 25 mmol/L MOPS, 100 mmol/L phenylmethylsulfonyl fluoride, and 20 mmol/L leupeptin, adjusted to pH 7.2. Lysis of tumor specimens was performed using Omni Tissue Homogenizer (TH; Omni International, Kennesaw, Ga.). Lysates were subjected to Western blot analysis using antibodies specific for p-EGFR (Tyr1173), EGFR, Akt, ERK, IGFBP3, IGF1R and actin (obtained from Santa Cruz Biotechnology, Santa Cruz, Calif.), and antibodies specific for p-ErbB2 (Tyr1221/1222), ErbB2, p-ErbB3 (Tyr1289), ErbB3, p-IGF1R (Tyr1135/1136), p-Akt (Ser473), p-ERK (Thr202/Tyr204), caspase-3 and PARP-1 (purchased from Cell Signaling Technology, Beverly, Mass.). Proteins were detected with an enhanced chemiluminescence Western blotting kit (Amersham Biosciences), according to the manufacturer's instructions.

Phopho-Receptor Tyrosine Kinase (RTK) Array Analysis

Cells were grown to confluence, then cell lysates were prepared by protein extraction. The RTK array experiment was performed according to manufacturer's instruction (RayBiotech, Norcross, Ga.) and as described previously (Chung J H, Rho J K, Xu X et al. Clinical and molecular evidences of epithelial to mesenchymal transition in acquired resistance to EGFR-TKIs. Lung Cancer 2011; 73: 176-182).

Xenograft Studies

To establish the PC-9/GR/WR xenograft model, female severe combined immunodeficiency (SCID) mice (18-20 g, 6 weeks of age) were purchased from Charles River Laboratories. All experimental procedures were conducted following a protocol approved by the Institutional Animal Care and Use committee of Asan Institute for Life Sciences (2015-02-062). Tumors were grown by implanting PC-9/GR/WR cells (1-5×10⁶ cells/0.1 mL) in 50% Matrigel (BD Biosciences), and subcutaneously injected into the right flank of animals.

Combination with WZ4002: Drug treatments, with control (10% 1-methyl-2-pyrrolidinone: 90% PEG-300, oral gavage), WZ4002 (30 mg/kg, oral gavage, 5 days a week), BI-IGF (100 mg/kg, i.p., 2 days a week), or WZ4002 plus BI-IGF, were started with 5 mice per group when tumor volumes reached 50-100 mm³.

Combination with HM61713: Drug treatments, with control (10% 1-methyl-2-pyrrolidinone: 90% PEG-300, oral gavage), HM61713 (200 and 400 mg/kg, oral gavage, 5 days a week), BI-IGF (100 mg/kg, i.p., 2 days a week), or HM61713 plus BI-IGF, were started with 7-10 mice per group when tumor volumes reached 150-250 mm³.

For evaluation of tumor sizes, the length (L) and the width (W) of each tumor were measured using calipers, and tumor volume (TV) was calculated as TV=(L×W²)/2. Body weights were measured every 3-4 days. At the end of the combination experiment with WZ4002, tumors from each group were homogenized for lysate preparation and analyzed by Western Blotting.

Results:

Activation of IGF1R signaling was identified as a mechanism of resistance to 3G-EGFR inhibitors in NSCLC. Phosphorylation of IGF1R and downstream pathway molecules were enhanced in PC-9/GR/WR NSCLC cells harboring acquired resistance to the EGFR inhibitor compound 2. An exemplary combination of the EGFR inhibitor compound 2 and the anti-IGF1/2 antibody BI-IGF led to recovery of sensitivity to compound 2 in vitro, i.e. potently inhibited the proliferation of PC-9/GR/WR cells, suppressed both EGFR and IGF1R signaling, and resulted in induction of apoptosis.

Further, the inhibition of IGF1R activity by treatment with the IGF1/2 neutralizing antibody BI-IGF overcame acquired resistance to compound 2 in PC-9/GR/WR tumor xenografts in vivo. Compared to treatment with compound 2 alone, the combination of compound 2 with BI-IGF resulted in substantially enhanced tumor growth inhibition, associated with pronounced inhibition of EGFR and IGF1R activity and signaling, and induction of apoptosis.

Cross-resistance of PC-9/GR/WR tumor xenografts to the 3G-EGFR inhibitor compound 1 (=HM61713) was observed. Combination with the IGF1/2 neutralizing antibody BI-IGF likewise overcame resistance to compound 1 in PC-9/GR/WR tumor xenografts in vivo. Treatment with the combination of compound 1, at both dose levels, and BI-IGF completely blocked tumor growth. There was a statistically significant difference in tumor growth inhibition between the combination groups versus control and monotherapy groups (P=0.000319 for 200 mg/kg compound 1 plus BI-IGF versus control, P=0.00000796634 for 200 mg/kg compound 1 plus BI-IGF versus BI-IGF, P=0.001691 for 200 mg/kg compound 1 plus BI-IGF versus 200 mg/kg compound 1, P=0.000334 for 400 mg/kg compound 1 plus BI-IGF versus control, P=0.000441395 for 400 mg/kg compound 1 plus BI-IGF versus BI-IGF and P=0.000829 for 400 mg/kg compound 1 plus BI-IGF versus 400 mg/kg compound 1). Combination treatments were well tolerated, i.e. did not cause significant body weight loss in mice.

The data support that a combination according to the present invention is useful for the herein-described therapeutic purposes, such as e.g. for treating NSCLC.

SEQUENCE LISTING

SEQ ID NO: 1
Ser Tyr Trp Met Ser
SEQ ID NO: 2
Ser Ile Thr Ser Tyr Gly Ser Phe Thr Tyr Tyr Ala
Asp Ser Val Lys
SEQ ID NO: 3
Asn Met Tyr Thr His Phe Asp Ser
SEQ ID NO: 4
Ser Gly Ser Ser Ser Asn Ile Gly Ser Asn Ser Val
Ser
SEQ ID NO: 5
Asp Asn Ser Lys Arg Pro Ser
SEQ ID NO: 6
Gln Ser Arg Asp Thr Tyr Gly Tyr Tyr Trp Val
SEQ ID NO: 7
Gln Val Glu Leu Val Glu Ser Gly Gly Gly Leu Val
Gln Pro Gly Gly Ser Leu Arg Leu Ser Cys Ala Ala
Ser Gly Phe Thr Phe Thr Ser Tyr Trp Met Ser Trp
Val Arg Gln Ala Pro Gly Lys Gly Leu Glu Leu Val
Ser Ser Ile Thr Ser Tyr Gly Ser Phe Thr Tyr Tyr
Ala Asp Ser Val Lys Gly Arg Phe Thr Ile Ser Arg
Asp Asn Ser Lys Asn Thr Leu Tyr Leu Gln Met Asn
Ser Leu Arg Ala Glu Asp Thr Ala Val Tyr Tyr Cys
Ala Arg Asn Met Tyr Thr His Phe Asp Ser Trp Gly
Gln Gly Thr Leu
SEQ ID NO: 8
Asp Ile Val Leu Thr Gln Pro Pro Ser Val Ser Gly
Ala Pro Gly Gln Arg Val Thr Ile Ser Cys Ser Gly
Ser Ser Ser Asn Ile Gly Ser Asn Ser Val Ser Trp
Tyr Gln Gln Leu Pro Gly Thr Ala Pro Lys Leu Leu
Ile Tyr Asp Asn Ser Lys Arg Pro Ser Gly Val Pro
Asp Arg Phe Ser Gly Ser Lys Ser Gly Thr Ser Ala
Ser Leu Ala Ile Thr Gly Leu Gln Ser Glu Asp Glu
Ala Asp Tyr Tyr Cys Gln Ser Arg Asp Thr Tyr Gly
Tyr Tyr Trp Val Phe Gly Gly Gly Thr Lys Leu Thr
Val Leu Gly
SEQ ID NO: 9
Gln Val Glu Leu Val Glu Ser Gly Gly Gly Leu Val
Gln Pro Gly Gly Ser Leu Arg Leu Ser Cys Ala Ala
Ser Gly Phe Thr Phe Thr Ser Tyr Trp Met Ser Trp
Val Arg Gln Ala Pro Gly Lys Gly Leu Glu Leu Val
Ser Ser Ile Thr Ser Tyr Gly Ser Phe Thr Tyr Tyr
Ala Asp Ser Val Lys Gly Arg Phe Thr Ile Ser Arg
Asp Asn Ser Lys Asn Thr Leu Tyr Leu Gln Met Asn
Ser Leu Arg Ala Glu Asp Thr Ala Val Tyr Tyr Cys
Ala Arg Asn Met Tyr Thr His Phe Asp Ser Trp Gly
Gln Gly Thr Leu Val Thr Val Ser Ser Ala Ser Thr
Lys Gly Pro Ser Val Phe Pro Leu Ala Pro Ser Ser
Lys Ser Thr Ser Gly Gly Thr Ala Ala Leu Gly Cys
Leu Val Lys Asp Tyr Phe Pro Glu Pro Val Thr Val
Ser Trp Asn Ser Gly Ala Leu Thr Ser Gly Val His
Thr Phe Pro Ala Val Leu Gln Ser Ser Gly Leu Tyr
Ser Leu Ser Ser Val Val Thr Val Pro Ser Ser Ser
Leu Gly Thr Gln Thr Tyr Ile Cys Asn Val Asn His
Lys Pro Ser Asn Thr Lys Val Asp Lys Lys Val Glu
Pro Lys Ser Cys Asp Lys Thr His Thr Cys Pro Pro
Cys Pro Ala Pro Glu Leu Leu Gly Gly Pro Ser Val
Phe Leu Phe Pro Pro Lys Pro Lys Asp Thr Leu Met
Ile Ser Arg Thr Pro Glu Val Thr Cys Val Val Val
Asp Val Ser His Glu Asp Pro Glu Val Lys Phe Asn
Trp Tyr Val Asp Gly Val Glu Val His Asn Ala Lys
Thr Lys Pro Arg Glu Glu Gln Tyr Asn Ser Thr Tyr
Arg Val Val Ser Val Leu Thr Val Leu His Gln Asp
Trp Leu Asn Gly Lys Glu Tyr Lys Cys Lys Val Ser
Asn Lys Ala Leu Pro Ala Pro Ile Glu Lys Thr Ile
Ser Lys Ala Lys Gly Gln Pro Arg Glu Pro Gln Val
Tyr Thr Leu Pro Pro Ser Arg Asp Glu Leu Thr Lys
Asn Gln Val Ser Leu Thr Cys Leu Val Lys Gly Phe
Tyr Pro Ser Asp Ile Ala Val Glu Trp Glu Ser Asn
Gly Gln Pro Glu Asn Asn Tyr Lys Thr Thr Pro Pro
Val Leu Asp Ser Asp Gly Ser Phe Phe Leu Tyr Ser
Lys Leu Thr Val Asp Lys Ser Arg Trp Gln Gln Gly
Asn Val Phe Ser Cys Ser Val Met His Glu Ala Leu
His Asn His Tyr Thr Gln Lys Ser Leu Ser Leu Ser
Pro Gly Lys
SEQ ID NO: 10
Asp Ile Val Leu Thr Gln Pro Pro Ser Val Ser Gly
Ala Pro Gly Gln Arg Val Thr Ile Ser Cys Ser Gly
Ser Ser Ser Asn Ile Gly Ser Asn Ser Val Ser Trp
Tyr Gln Gln Leu Pro Gly Thr Ala Pro Lys Leu Leu
Ile Tyr Asp Asn Ser Lys Arg Pro Ser Gly Val Pro
Asp Arg Phe Ser Gly Ser Lys Ser Gly Thr Ser Ala
Ser Leu Ala Ile Thr Gly Leu Gln Ser Glu Asp Glu
Ala Asp Tyr Tyr Cys Gln Ser Arg Asp Thr Tyr Gly
Tyr Tyr Trp Val Phe Gly Gly Gly Thr Lys Leu Thr
Val Leu Gly Gln Pro Lys Ala Ala Pro Ser Val Thr
Leu Phe Pro Pro Ser Ser Glu Glu Leu Gln Ala Asn
Lys Ala Thr Leu Val Cys Leu Ile Ser Asp Phe Tyr
Pro Gly Ala Val Thr Val Ala Trp Lys Gly Asp Ser
Ser Pro Val Lys Ala Gly Val Glu Thr Thr Thr Pro
Ser Lys Gln Ser Asn Asn Lys Tyr Ala Ala Ser Ser
Tyr Leu Ser Leu Thr Pro Glu Gln Trp Lys Ser His
Arg Ser Tyr Ser Cys Gln Val Thr His Glu Gly Ser
Thr Val Glu Lys Thr Val Ala Pro Thr Glu Cys Ser

1. A method of treating and/or preventing an oncological or hyperproliferative disease, comprising administering to a patient in need thereof a therapeutically effective amount of a 3G-EGFR inhibitor selected from the group consisting of compound 1 and compound 2—or a pharmaceutically acceptable salt thereof— 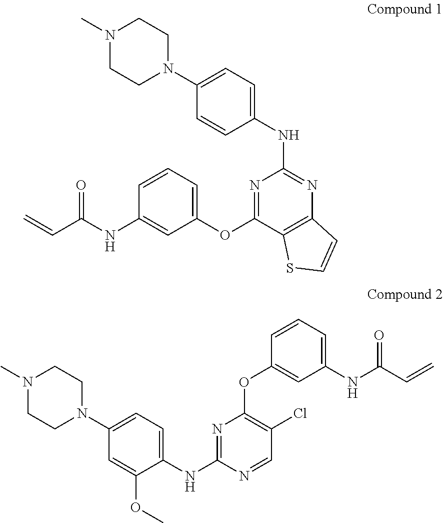 [SMILES: C=CC(=O)Nc1cccc(Oc2nc(Nc3ccc(N4CCN(C)CC4)cc3)nc3ccsc23)c1 ; C=CC(=O)Nc1cccc(Oc2nc(Nc3ccc(N4CCN(C)CC4)cc3OC)ncc2Cl)c1]
and an anti-IGF antibody, wherein said antibody molecule has heavy chain CDRs comprising the amino acid sequences of SEQ ID NO:1 (CDR1), SEQ ID NO:2 (CDR2) and SEQ ID NO:3 (CDR3) and has light chain CDRs comprising the amino acid sequences of SEQ ID NO:4 (CDR1), SEQ ID NO:5 (CDR2) and SEQ ID NO:6 (CDR3).

2. The method according to claim 1, wherein the 3G-EGFR inhibitor is administered simultaneously, concurrently, sequentially, successively, alternately or separately with the anti-IGF antibody.

3. A method of treating and/or preventing an oncological disease, the method comprising administering to a patient in need thereof an anti-IGF antibody, having heavy chain CDRs comprising the amino acid sequences of SEQ ID NO:1 (CDR1), SEQ ID NO:2 (CDR2) and SEQ ID NO:3 (CDR3) and having light chain CDRs comprising the amino acid sequences of SEQ ID NO:4 (CDR1), SEQ ID NO:5 (CDR2) and SEQ ID NO:6 (CDR3), in combination with a 3G-EGFR inhibitor selected from the group consisting of compound 1 and compound 2—or a pharmaceutically acceptable salt thereof

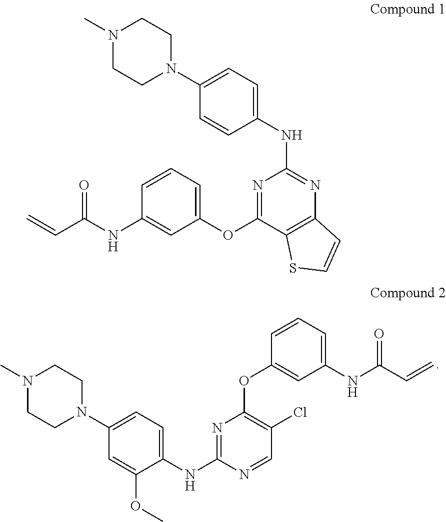 [SMILES: C=CC(=O)Nc1cccc(Oc2nc(Nc3ccc(N4CCN(C)CC4)cc3)nc3ccsc23)c1 ; C=CC(=O)Nc1cccc(Oc2nc(Nc3ccc(N4CCN(C)CC4)cc3OC)ncc2Cl)c1]

4. The method according to claim 3, wherein the anti-IGF antibody is administered simultaneously, concurrently, sequentially, successively, alternately or separately with the 3G-EGFR inhibitor.

5. A pharmaceutical composition comprising a 3G-EGFR inhibitor selected from the group consisting of compound 1 and compound 2—or a pharmaceutically acceptable salt thereof— 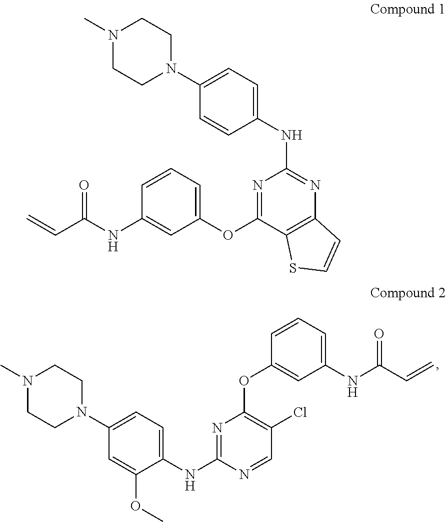 [SMILES: C=CC(=O)Nc1cccc(Oc2nc(Nc3ccc(N4CCN(C)CC4)cc3)nc3ccsc23)c1 ; C=CC(=O)Nc1cccc(Oc2nc(Nc3ccc(N4CCN(C)CC4)cc3OC)ncc2Cl)c1]
an anti-IGF antibody, wherein said antibody molecule has heavy chain CDRs comprising the amino acid sequences of SEQ ID NO:1 (CDR1), SEQ ID NO:2 (CDR2) and SEQ ID NO:3 (CDR3) and has light chain CDRs comprising the amino acid sequences of SEQ ID NO:4 (CDR1), SEQ ID NO:5 (CDR2) and SEQ ID NO:6 (CDR3), and, optionally, one or more pharmaceutically acceptable carriers, excipients and/or vehicles.

6. A kit comprising a first pharmaceutical composition comprising a 3G-EGFR inhibitor selected from the group consisting of compound 1 and compound 2—or a pharmaceutically acceptable salt thereof— 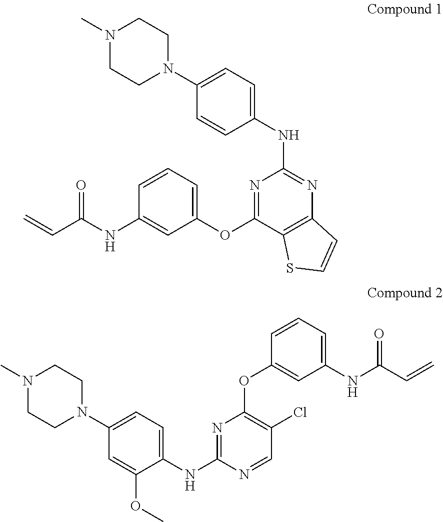 [SMILES: C=CC(=O)Nc1cccc(Oc2nc(Nc3ccc(N4CCN(C)CC4)cc3)nc3ccsc23)c1 ; C=CC(=O)Nc1cccc(Oc2nc(Nc3ccc(N4CCN(C)CC4)cc3OC)ncc2Cl)c1]
and optionally one or more pharmaceutically acceptable carriers, excipients and/or vehicles; and
a second pharmaceutical composition comprising an anti-IGF antibody, wherein said antibody molecule has heavy chain CDRs comprising the amino acid sequences of SEQ ID NO:1 (CDR1), SEQ ID NO:2 (CDR2) and SEQ ID NO:3 (CDR3) and has light chain CDRs comprising the amino acid sequences of SEQ ID NO:4 (CDR1), SEQ ID NO:5 (CDR2) and SEQ ID NO:6 (CDR3), and, optionally, one or more pharmaceutically acceptable carriers, excipients and/or vehicles.

7. The method according to claim 1, wherein the 3G-EGFR inhibitor is compound 1.

8. The method according to claim 1, wherein the antibody molecule has a variable heavy chain comprising the amino acid sequence of SEQ ID NO:7 and a variable light chain comprising the amino acid sequence of SEQ ID NO:8.

9. The method according to claim 1, wherein the antibody molecule has a heavy chain comprising the amino acid sequence of SEQ ID NO:9, and a light chain comprising the amino acid of SEQ ID NO:10.

10. The method according to claim 1, wherein the oncological disease to be treated is cancer harboring one or more EGFR mutation.

11. The method according to claim 10, wherein at least one EGFR mutation is selected from Del19 (deletion in exon 19), L858R and T790M.

12. The method according to claim 11, wherein the at least one EGFR mutation is Del19.

13. The method according to claim 11, wherein the at least one EGFR mutation is L858R.

14. The method according to claim 11, wherein the at least one EGFR mutation is T790M.

15. The method according to claim 11, wherein the cancer harbors at least two EGFR mutations selected from the group consisting of Del19/T790M and L858R/T790M.

16. The method according to claim 10, wherein the cancer is non-small cell lung cancer (NSCLC).
